(19) United States
(12) Patent Application Publication
(10) Pub. No.: US 2014/0261579 A1
(43) Pub. Date: Sep. 18, 2014

(54) APPARATUS FOR FLUSHING ANGLED WINDOW OF ENDOSCOPE

(71) Applicant: Acclarent, Inc., (US)

(72) Inventors: Thomas R. Jenkins, Alameda, CA (US); Eric Goldfarb, Belmont, CA (US); Fletcher T. Wilson, San Francisco, CA (US); Jessica Chan, Sunnyvale, CA (US)

(21) Appl. No.: 13/804,740

(22) Filed: Mar. 14, 2013

(51) Int. Cl. A61B 1/12

(52) U.S. Cl. CPC A61B 1/123
USPC 134/166 C

(57) ABSTRACT

An endoscope washing assembly includes a hub and a shaft. The hub has an inner member and an outer member coupled to a fluid source. The shaft extends distally from the hub. The shaft includes a distal portion, a fluid channel, and a lumen. The distal portion of the shaft corresponds with a distal portion of an endoscope. The fluid channel extends from the hub to the distal portion of the shaft and provides fluid communication with a fluid source. The lumen extends through the shaft and is configured to receive the endoscope. A boss structure is positioned within the shaft and is configured to maintain a radial position of an endoscope relative to the first fluid channel. The shaft may provide both saline and suction to the distal end of the endoscope.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Menlo Park, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein. An example of such an endoscope is the Acclarent Cyclops™ Multi-Angle Endoscope by Acclarent, Inc. of Menlo Park, Calif.

In view of the foregoing, it may be desirable to provide a washing system for use with the endoscope to maintain visualization within an anatomical passageway. While several systems and methods have been made and used to wash an endoscope for use in an anatomical passageway, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a side view of an exemplary dilation catheter system;

FIG. 2 depicts a perspective view of an endoscope for use with the dilation catheter system of FIG. 1;

FIG. 3 depicts a cross sectional view of a distal end of the endoscope of FIG. 2, showing viewing ranges of the endoscope;

FIG. 4 depicts a side view of an exemplary washing system for use with the endoscope of FIG. 2;

FIG. 5 depicts a side view of the washing system of FIG. 4 coupled with the endoscope of FIG. 2;

FIG. 6 depicts a perspective view of a hub of the washing system of FIG. 4;

FIG. 7 depicts a cross sectional view of the hub of FIG. 6;

FIG. 8 depicts a perspective view of a distal end of the washing system of FIG. 4;

FIG. 9 depicts an end view of the distal end of the washing system of FIG. 4;

FIG. 10 depicts a side view of the distal end of the washing system of FIG. 4, showing an endoscope inserted within the washing system;

FIG. 11 depicts an end view of the distal end of the washing system of FIG. 4, showing an endoscope inserted within the washing system;

FIG. 12 depicts a side view of another exemplary washing system for use with the endoscope of FIG. 2;

FIG. 13 depicts an end view of a distal end of the washing system of FIG. 12;

FIG. 14 depicts an end view of the distal end of the washing system of FIG. 12, showing an endoscope inserted within the washing system;

FIG. 15 depicts a perspective view of a distal end of another exemplary washing system for use with the endoscope of FIG. 2;

FIG. 16 depicts an end view of the distal end of the washing system of FIG. 15, showing an endoscope inserted within the washing system;

FIG. 17A depicts a cross sectional view of an exemplary actuator of a washing system in a closed position;

FIG. 17B depicts a cross sectional view of the actuator of FIG. 17A in an open position;

FIG. 18 depicts a side view of another exemplary washing system for use with the endoscope of FIG. 2;

FIG. 19 depicts a perspective view of an exemplary foot pedal to actuate the washing system of FIG. 18;

FIG. 20A depicts a side elevational view of the foot pedal of FIG. 19 in a non-actuated state, with a bulb and tube shown in cross-section;

FIG. 20B depicts a side elevational view of the foot pedal of FIG. 19 in an actuated state, with a bulb and tube shown in cross-section;

FIG. 21A depicts a front elevational view of the foot pedal of FIG. 19 in the non-actuated state, showing the bulb in a non-compressed state;

FIG. 21B depicts a front elevational view of the foot pedal of FIG. 19 in the actuated state, showing the bulb in a compressed state;

FIG. 22A depicts a rear elevational view of the foot pedal of FIG. 19 in the non-actuated state, showing the tube in a closed state;

FIG. 22B depicts a rear elevational view of the foot pedal of FIG. 19 in the actuated state, showing the tube in an open state;

FIG. 23A depicts a front elevational view of another exemplary foot pedal to actuate the washing system of FIG. 19, showing a second pedal in a non-actuated state;

FIG. 23B depicts a front elevational view of the foot pedal of FIG. 22A, showing the second pedal in an actuated state; and

FIG. 24 depicts a perspective view of the endoscope of FIG. 2 and the washing system of FIG. 4 coupled with an exemplary button valve system.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Dilation Catheter System

FIG. 1 shows an exemplary dilation catheter system (1), which may be used to dilate the ostium of a paranasal sinus; or to dilate some other anatomical passageway (e.g., within the ear, nose, or throat, etc.). Dilation catheter system (1) of this example comprises a dilation catheter (10), a dilator (14), a guide catheter (70), and an inflator (50). System (1) may be configured in accordance with at least some of the teachings of U.S. Patent Pub. No. 2011/0004057, entitled “Systems and Methods for Transnasal Dilation of Passageways in the Ear, Nose or Throat,” published Jan. 6, 2011, the disclosure of which is incorporated by reference herein. In some versions, at least part of system (1) is configured similar to the Relieva® Spin Balloon Sinuplasty™ System by Acclarent, Inc. of Menlo Park, Calif. Dilator (14) of the present example is coupled to a distal end of dilation catheter (10), and inflator (50) is coupled to a proximal portion of dilation catheter (10), such that dilation catheter (10) provides fluid communication between inflator (50) and dilator (14). Dilation catheter (10) is slidably positioned through guide catheter (70).

In a dilation procedure, guide catheter (70) may first be positioned near the targeted anatomical passageway. Guide catheter (70) is initially inserted into the nose of the patient and is advanced to a position that is within or near the ostium to be dilated. After guide catheter (70) has been positioned, the operator may insert the distal end of a guidewire (GW) into the proximal end of guide catheter (70) and may advance the guidewire (GW) through guide catheter (70) such that a distal portion of the guidewire (GW) passes through the sinus ostium (SO) and becomes coiled within the sinus cavity. Thereafter, the proximal end of the guidewire (GW) is inserted into the distal end of dilation catheter (10), and dilation catheter (10) (with dilator (14) in a non-expanded state) is advanced over the guidewire (GW) and through guide catheter (70) to a position where dilator (14) is positioned within the sinus ostium (SO), or other targeted anatomical passageway.

After dilator (14) has been positioned within the ostium, dilator (14) may be inflated, thereby dilating the ostium. To inflate dilator (14), plunger (54) may be actuated to push saline from inflator (50) through dilation catheter (10) into dilator (14). The transfer of fluid expands dilator (14) to an expanded state to open or dilate the targeted anatomical passageway. Dilator (14) may be inflated to a volume size to achieve about 10 to about 12 atmospheres. Dilator (14) may be held at this volume for a few seconds to sufficiently open the ostium or targeted anatomical passageway. Dilator (14) may then be deflated (or returned to a non-expanded state) by actuating plunger (54) of inflator (50) to bring the saline back to inflator (50). Dilator (14) may be repeatedly inflated and deflated in different ostia and/or other targeted anatomical passageways. Thereafter, dilation catheter (10), guidewire (GW), and guide catheter (70) may be removed from the patient.

II. Exemplary Endoscope

In some uses of system (1) shown in FIG. 1, an endoscope may be placed in the ear, nose, or throat to view all or part of the procedure. For instance, the successful positioning of dilator (14) and dilation of the ostium may be confirmed visually using an endoscope. FIG. 2 shows an exemplary endoscope (110) for use with the dilation catheter system of FIG. 1 or in any other suitable type of context. In some versions, endoscope (110) may be configured in accordance with at least some of the teachings of U.S. Pub. No. 2010/0030031, entitled “Swing Prism Endoscope,” published Feb. 4, 2010, the disclosure of which is incorporated by reference herein. Endoscope (110) of the present example comprises an elongate shaft (130) with a distal end (170) and a proximal end (171). Proximal end (171) of shaft (130) is attached to handle (152). Distal end (170) of endoscope (110) may be placed within the desired anatomical passageway to provide visualization to the user. The user may visualize images through an eyepiece (150) or on a visualization system (e.g., camera and viewing screen, etc.) coupled with eyepiece (150). While endoscope (110) is described herein as being used in combination with system (1), it should be understood that endoscope (110) may be used in a variety of other procedures in combination with a variety of other instruments/devices.

A. Exemplary Shaft

As shown in FIG. 3, shaft (130) houses an image fiber bundle or optic fibers (154) that extend coaxially through the center of shaft (130). Shaft (130) may also include light transmitting fibers disposed about the periphery of optic fibers (154). In some versions, shaft (130) is a braided polyimide sheathing that has a maximum outer diameter of about 0.0375 inches and a length of about two feet. Endoscope shaft (130) may have an outer diameter of approximately 4 mm and a working length of about 175 mm. Shaft (130) has rounded surfaces thus making the assembly atraumatic in use. Optic fibers (154) are made up of 10,000 thin image fibers, and the light transmitting fibers are illumination fibers with a diameter of between about 0.008 and 0.020 inches, with a minimum lux of about 10,000. In other versions, endoscope (110) uses rod lens technology instead of image fiber bundles. Of course, any of the above-noted parameters may be varied as desired.

As shown in FIG. 3, distal end (170) of shaft (130) comprises a swing prism (172), a lens (174), an actuator (178), and a resilient member (176). Swing prism (172) is mounted for rotation between resilient member (176) and actuator (178). In particular, swing prism (172) is pivotable about an axis (171), which is transverse to the longitudinal axis of shaft (130). Actuator (178) may comprise a wire which extends from distal portion (170) of endoscope (110) to a proximal portion. Actuator (178) is driven by rotation of proximal dial (108) relative to handle (152). Actuator (178) may be attached to a sliding member or configured to be taken up by a rotating dial (not shown). Suitable actuator (178) configurations will be apparent to one with ordinary skill in the art in view of the teachings herein. Actuator (178) may be used to rotate swing prism (172) within distal end (170). Swing prism (172) is positioned adjacent to a window (175), which may be formed by glass, crystal, plastic, and/or any other suitable material. A self-focusing lens (174) is positioned between swing prism (172) and fiber bundle (154). Images may be captured and received through window (175) and transmitted through swing prism (172) and self-focusing lens (174) to image fiber bundle (154). In the present example, swing prism (172) provides a seventy degree field of view throughout a viewing range of zero degrees to ninety five degrees by manipulating actuator (178). Alternatively, any other suitable viewing range may be provided.

Distal end (170) of endoscope shaft (130) is shown with angular measurements in FIG. 3. In describing FIG. 3, “field of view” means the angular width/height viewed at any one time via endoscope (110), “direction of view” means the direction in which the center of view or line of sight is pointing at any one time (also can be called the “degree of view” as in “variable degree of view endoscope”) and “total range of view” means the total angular distance across which endoscope (110) can view when the swing prism is moved from one extreme direction of view to the opposite extreme direction of view. The angles referred to are in relation to the longitudinal axis of endoscope shaft (130), which is the zero angle.

In the present example, endoscope (110) has a range of directions of view from about −5° to about 150°, such as from about 0° to about 120°, from about 5° to about 100°, or from about 10° to about 90°. In some versions, endoscope (110) has a field of view from about 50° to about 100°, such as from about 60° to about 70°. From the ranges of the directions of view and the fields of view, the total ranges of view may be determined. For example, if endoscope (110) has directions of view ranging from about 5° to about 100° and a field of view of about 60°, the total range of view would be from about −25° to about 130°. If the ranges of directions of view were instead from about 0° to about 120° and the field of view were about 60°, then the total range of view would be from about −30° to about 150°. Endoscope (110) may have any of a number of different combinations and ranges of directions of view, fields of view and total ranges of view.

The images collected by image fiber bundle (154) may be transmitted to a monitor (described below) to thereby provide the operator with visualization of the procedure being performed. In some versions, endoscope (110) is compatible with a 300 Watt Xenon source and is configured with a universal light guide connector, thus making the assembly useable with conventionally available devices.

B. Exemplary Handle

As shown in FIG. 2, proximal end (171) of shaft (130) is coupled to a handle (152). Handle (152) comprises dials (108, 112), and light post (109). A proximal dial (108) is disposed on handle (152) of endoscope (110) for controlling the rotation of swing prism (172). Proximal dial (108) has a circular configuration and includes ridges that provide leverage for turning proximal dial (108) to a desired position. Handle (152) includes indicia (107) adjacent the proximal dial (108) to provide information relative to the direction of view of swing prism (172). As shown, indicia (107) adjacent to proximal dial (108) indicates the direction of view of swing prism (172) anywhere from 0 degrees to 180 degrees. A distal dial (112) is disposed on handle (152) to control rotation of endoscope shaft (130) relative to handle (152), about the longitudinal axis of shaft (130). A marker (114) is shown on distal dial (112) to indicate the relative position of endoscope shaft (130) and window (175). Rotating distal dial (112) allows endoscope (110) to view its surroundings in a full three-hundred and forty degrees of rotation. Having a rotating distal dial (112) that rotates endoscope shaft (130) without rotating the entire handle (152) may allow for rotation of endoscope shaft (130) without rotating post (109). Post (109) extends from handle (152) to connect wiring for optic fibers (154).

III. Exemplary Washing System

During procedures, endoscope (110) may lose visual clarity because of debris, blood, and/or mucus adhering to distal end (170), and particularly to window (175). Surgeons or users may remove endoscope (110) from the patient frequently to clean window (175) and other portions of distal end (170). Accordingly, it may be desirable to provide a washing system to clear debris from window (175) and perhaps other portions of distal end (170) of endoscope (110) without having to remove endoscope (110) from the patient. An actuation assembly may be provided with the washing system to selectively clear debris from window (175) and perhaps other portions of distal end (170) of endoscope (110). The examples below include merely illustrative versions of a washing system that may be readily incorporated to a variation of endoscope (110). The below washing features and techniques may be readily combined with the wiping features and techniques of U.S. Patent App. Ser. No. [ATTORNEY DOCKET NO. ACC5052USPSP.0600456], entitled “Apparatus for Wiping Angled Window of Endoscope,” filed even herewith, the disclosure of which is incorporated by reference herein.

A. Exemplary Washing Assembly

FIGS. 4-5 show an exemplary washing assembly (210) that may be coupled with endoscope (110). Washing assembly (210) comprises a shaft (230) having a distal end (232) and a proximal end (271). Shaft (230) may comprise steel, plastic (e.g., Nylon, Pebax, Peek, etc.), and/or any other suitable materials. Suitable shaft (230) materials will be apparent to one with ordinary skill in the art in view of the teachings herein. Proximal end (271) of shaft (230) is coupled to hub (270). As shown in FIG. 5, washing assembly (210) is slidably positioned over endoscope (110). Washing assembly (210) may be releasably coupled with endoscope (110), or washing assembly (201) may be fixedly secured with endoscope (110). Shaft (130) of endoscope (110) is inserted through an opening of hub (270) and into shaft (230) of washing assembly (210). A distal end of handle (152) engages inner hub (270) of washing assembly (210) such that endoscope (110) is inserted within washing assembly (210) until distal end (170) of endoscope (110) substantially aligns with distal end (232) of washing assembly (210). Washing assembly (210) is operable to substantially clear window (175) of debris, thereby improving the view provided through endoscope (110).

1. Exemplary Hub

As shown in FIG. 6, hub (270) comprises an opening (280) to receive endoscope (110). Opening (280) comprises recesses (282) extending outwardly from opening (280). The proximal end of handle (152) of endoscope (152) has protrusions corresponding to recesses (282). Accordingly, when endoscope (110) is inserted within washing assembly (210), the protrusions of handle (152) engage recesses (282) of hub (270). This aligns hub (270) with handle (152) such that distal ends (170, 232) are angularly aligned; and such that when distal dial (112) is rotated, endoscope shaft (130) and washing assembly shaft (230) rotate unitarily. Although two recesses (282) are shown, any number of recesses (282) may be used. Further, hub (270) may be coupled to handle (152) and/or shaft (130) via a friction fit or using any other suitable types of features or relationships. Various suitable relationships between hub (270) and handle (152) and/or hub (270) and shaft (130) will be apparent to one with ordinary skill in the art in view of the teachings herein.

Hub (270) further comprises a fixed mount (272) positioned around hub (270). Hub (270) may rotate freely relative to fixed mount (272). For instance, hub (270) may be coupled with fixed mount (272) by one or more bushings, bearings, and/or other features configured to facilitate rotation. As shown in FIG. 7, fixed mount (272) comprises a fluid inlet (274) with an opening (284). Fluid inlet (274) is coupled with a fluid source to provide fluid through opening (284) to fluid inlet (274). The fluid source may provide saline, air, pressurized gas, etc. The fluid provided through fluid inlet (274) is communicated through opening (284) of fluid inlet (274) to shaft (230) via an opening (273) in hub (270). A pair of O-rings (276) fluidly seal fixed mount (272) and hub (270). When endoscope (110) is loaded within washing assembly (210), another O-ring (277) fluidly seals hub (270) with shaft (130) of endoscope (110). O-ring (277) also provides enough friction to substantially retain shaft (130) relative to hub (270) in the longitudinal direction. Because hub (270) is free to rotate within fixed mount (272), hub (270) and shaft (230) may be rotated without entangling any tubing connecting fixed mount (272) to the fluid source. It should be understood that rotation of hub (270) within fixed mount (272) does not compromise the seals provided by o-rings (276) or the communication of fluid from inlet (274) through opening (273). The relationship between hub (270) and fixed mount (272) thus provides a hydraulic slip ring or fluid rotary union. In some other versions, hub (270) and fixed mount (272) are unitary, such that hub (270) is not rotatable relative to fixed mount (272).

2. Exemplary Distal End with a Single Fluid Lumen

FIGS. 8-11 show distal end (232) of washing assembly shaft (230) in greater detail. Distal end (232) comprises an opening (233) extending through shaft (230) and protrusions (234), as shown in FIG. 9. Protrusions (234) comprise ridges (236) extending inwardly to form a recess (235) on a top portion of opening (233), as shown in FIG. 9. In the present example, ridges (236) are formed as unitary features of shaft (230). By way of example only, ridges (236) may be formed with a die that is used to extrude shaft (230); or in a molding process. As another merely illustrative example, ridges (236) may be formed by shims that are added to the interior of a cylindrical shaft (230). As yet another merely illustrative example, ridges (236) may be formed by bending protrusions (234) inwardly. Ridges (236) may extend along the full length of shaft (230) or along just part of the length of shaft (236). For instance, ridges (236) may be located in just a distal portion of the length of shaft (230), without extending proximally from that distal portion. Other suitable ridge (236) configurations will be apparent to one with ordinary skill in the art in view of the teachings herein. Although two ridges (236) are shown, any number of ridges (236) may be used.

When endoscope (110) is loaded within washing assembly (210), distal end (170) of endoscope (110) substantially aligns with distal end (232) of washing assembly (210), as shown in FIG. 10. Distal end (170) protrudes just slightly from shaft (230) such that shaft (230) does not interfere with the lighting and/or visualization at distal end (170) of endoscope (110). Shaft (130) of endoscope (110) is positioned within opening (233) of shaft (230). Opening (233) is sized to correspond to the size of shaft (130). Ridges (236) engage distal end (170) of endoscope (110), as shown in FIG. 11. Recess (235) extends above distal end (170) to define a fluid lumen (240) between shaft (230) and shaft (130). Accordingly, fluid is communicated from the fluid source, through hub (270) to distal end (232) via fluid lumen (240). It should be understood that ridges (236) maintain the radial positioning of shaft (130) within shaft (230), such that the longitudinal axis of shaft (130) remains parallel to the longitudinal axis of shaft (230); and such that the longitudinal axis of shaft (130) remains parallel to the longitudinal axis of lumen (240). Thus, ridges (236) act as bosses ensuring that the size and configuration of lumen (240) remains consistent during use of washing assembly (210) with endoscope (110).

In some versions, the fluid communicated through lumen (240) is saline or other biocompatible material. Because fluid lumen (240) is positioned on a top portion of distal end (170) of endoscope (110), saline communicated through fluid lumen (240) may cascade down distal end (170) to wash distal end (170) of endoscope (110). The saline may wash over window (175) substantially tangentially. In the present example, the position and configuration of fluid lumen provides a substantially laminar flow of saline or other liquid across window (175). Thus may be attributed at least in part to the fact that fluid lumen (240) longitudinally terminates just proximal to window (175). In addition or in the alternative, the laminar flow of saline or other liquid across window (175) may be attributed at least in part to the angular extent of fluid lumen (240) (e.g., where it terminates laterally). For instance, in some versions fluid lumen (240) extends angularly from approximately a 10 o'clock position to approximately a 2 o'clock position. In addition or in the alternative, the laminar flow of saline or other liquid across window (175) may be attributed at least in part to the fact that window (175) defines a curve that includes one or more chords that extend obliquely relative to the longitudinal axes of shaft (130), shaft (230), and fluid lumen (240). In other words, the flow of liquid over a window might not otherwise be laminar if the window is oriented substantially perpendicular to the longitudinal axes of shaft (130), shaft (230), and fluid lumen (240). It should be understood that a laminar flow of liquid over window (175) may provide the most efficient and effective washing of window (175), as compared to a non-laminar liquid flow.

The saline or other liquid that is communicated through fluid lumen (240) may be stored in a fluid reservoir or saline bag located some distance away from endoscope (110). The reservoir may be connected to washing assembly (210) via a flexible or rigid tube. Alternatively, the reservoir may be located around shaft (230) of washing assembly (210) proximal to distal end (232). A pumping system may be provided to move the saline or other liquid from the reservoir through fluid lumen (240). By way of example only, a reversible peristaltic pump may be used to communicate saline to lumen (240) and/or to draw saline from lumen (240). Alternatively, a manual pump (e.g., bulb, bellows, syringe, etc.) may be used or some other type of powered pump may be used. In addition or in the alternative, the reservoir may be positioned above washing assembly (210) such that gravity is able to drive the saline from the reservoir through fluid lumen (240). Other pumping configurations will be apparent to one with ordinary skill in the art in view of the teachings herein. The flow of saline or other liquid may be continuously provided to distal end (170); or may be selectively activated.

In some versions, suction may be provided through fluid lumen (240) to clear distal end (170) of endoscope (110) (e.g., particularly window (175)) of debris. Fluid lumen (240) may be connected to a low pressure source to provide a vacuum (i.e., suction) to remove debris from distal end (170). The low pressure source may be an electrically powered vacuum pump, a syringe, or a piston, etc. to manually create a pressure difference, or some other device. Suitable low pressure source configurations will be apparent to one with ordinary skill in the art in view of the teachings herein. Fluid lumen (240) may also extend around a greater circumferential range of distal end (232) to provide a greater amount of suction. The suction may be continuously provided to distal end (170) or may be selectively activated.

In some versions, pressurized dry gas may be provided through fluid lumen (240) to clear distal end (170) of endoscope (110) of debris. The gas may act to move an obscuring substance from distal end (170) by creating a high flow channel over and/or on distal end (170). Argon, nitrogen, or any dry gas that is pressurized may be used. The gas may be transported from a pressurized reservoir through fluid lumen (240). The gas may be continuously provided to distal end (170) or may be selectively activated. Washing assembly (210) may alternate between saline, suction, and/or pressurized gas through fluid lumen (240) during a washing process. For instance, saline may be provided through fluid lumen (240) then suction; or pressurized gas then suction; or any other suitable combination.

3. Exemplary Distal End with a Double Fluid Lumen

Another exemplary washing assembly (310) is shown in FIG. 12. Washing assembly (310) is similar to washing assembly (210), except that washing assembly (310) comprises a fluid source (382) and a suction source (384). Fixed mount (372) of washing assembly (310) comprises a first fluid inlet (374) coupled to fluid source (382) and a second fluid inlet (376) coupled to suction source (384). In some other versions, fixed mount (372) just has one inlet (e.g., similar to inlet (274)). In some such versions, fluid source (382) and suction source (384) both have outputs coupled with the same T-fitting, Y-fitting, or other type of junction; and the output for that T-fitting, Y-fitting, or other type of junction is coupled with the single inlet of fixed mount (372).

In the present example, control unit (380) is coupled to both the fluid source (382) and the suction source (384). Control unit (380) may be used to selectively actuate first fluid source (382) and/or suction source (384). By way of example only, fluid source (382) may comprise a reversible peristaltic pump, some other kind of powered pump, and/or any suitable type of manual pump (e.g., bulb, bellows, syringe, etc.). Suction source (384) may also comprise a reversible peristaltic pump, some other kind of powered pump, and/or any suitable type of manual pump (e.g., bulb, bellows, syringe, etc.). In some versions, a single pump may be used to drive fluid into fixed mount (372) via first fluid inlet (374) and draw fluid from fixed mount (372) via second fluid inlet (376). By way of example only, first fluid inlet (374) may be coupled with the outlet of a peristaltic pump while second fluid inlet (374) is coupled with the inlet of the same peristaltic pump. Such a pumping system may also comprise an additional inlet coupled with a saline bag or other fluid source, in order to ensure that enough fluid continues to flow through the system. Other suitable arrangements will be apparent to those of ordinary skill in the art in view of the teachings herein.

As shown in FIGS. 13-14, distal end (332) of washing assembly (310) comprises an opening (333). Ridges (336) extend inwardly from shaft (330) into opening (333). In the present example, ridges (336) are formed as unitary features of shaft (330). By way of example only, ridges (336) may be formed with a die that is used to extrude shaft (330); or in a molding process. As another merely illustrative example, ridges (336) may be formed by shims that are added to the interior of a cylindrical shaft (330). As yet another merely illustrative example, ridges (336) may be formed by bending protrusions (234) inwardly. Ridges (336) may extend along the full length of shaft (330) or along just part of the length of shaft (336). For instance, ridges (336) may be located in just a distal portion of the length of shaft (330), without extending proximally from that distal portion. Other suitable ridge (336) configurations will be apparent to one with ordinary skill in the art in view of the teachings herein.

Ridges (336) extend less than halfway around shaft (330) to form a top recess (335) and a bottom recess (337). When endoscope (110) is loaded into washing assembly (310), as shown in FIG. 14, distal end (170) engages ridges (336). Ridges (336) are sized to correspond to distal end (170). Top recess (335) extends above distal end (170) to form a first fluid lumen (340) between a top portion of distal end (170) and shaft (330). Bottom recess (337) extends below distal end (170) to form a second fluid lumen (342) between a bottom portion of distal end (170) and shaft (330). It should be understood that ridges (336) maintain the radial positioning of shaft (130) within shaft (330), such that the longitudinal axis of shaft (130) remains parallel to the longitudinal axis of shaft (330); and such that the longitudinal axis of shaft (130) remains parallel to the longitudinal axes of lumens (340, 342). Thus, ridges (236) act as bosses ensuring that the size and configuration of lumens (340, 342) remains consistent during use of washing assembly (310) with endoscope (110).

In the present example, first fluid lumen (340) is in fluid communication with first fluid inlet (374); while second fluid lumen (342) is in fluid communication with second fluid inlet (376). Thus, first fluid lumen (340) is configured to communicate fluid from fluid source (382); while second fluid lumen (342) is configured to draw suction toward suction source (384). First fluid lumen (340) and second fluid lumen (342) are fluidly isolated from each other along the length of shaft (330) in the present example. It should also be understood that shaft (330) and hub (370) are both rotatable together relative to fixed mount (372) without affecting the pneumatic/hydraulic state of inlets (374, 376) or lumens (340, 342). Thus, fixed mount (372) and hub (370) cooperate to act as a hydraulic slip ring or fluid rotary union with two channels that are isolated relative to each other. Various suitable ways in which fixed mount (372) may be configured to provide such functionality will be apparent to those of ordinary skill in the art in view of the teachings herein. Although two lumens (340, 342) are shown, any number of lumens may be used. Other fluid lumen (340, 342) configurations will be apparent to one with ordinary skill in the art in view of the teachings herein. By way of example only, in versions where fluid lumens (340, 342) only extend along a portion of the length of shaft (330), fluid lumens (340, 342) are not fluidly isolated from each other. Such versions may be combined with versions where fixed mount (372) has just one fluid inlet. Thus, when fluid is communicated from fluid source (382) in such versions, that fluid will be expelled through both lumens (340, 342). Similarly, when suction is communicated from suction source (384) in such versions, that suction will be provided at the distal ends of both lumens (340, 342).

It should be understood that the fluid from fluid source (382) may comprise any suitable liquid (e.g., saline, etc.) and/or gas. For instance, first lumen (340) may provide saline and second lumen (342) may provide suction; or first lumen (340) may provide pressurized gas and second lumen (342) may provide suction. Such fluid communication may be provided simultaneously or in a series (e.g. saline may be provided then suction; or pressurized gas may be provided then suction). Various suitable combinations and algorithms will be apparent to those of ordinary skill in the art in view of the teachings herein. It should also be understood that first lumen (340) may provide a laminar flow of fluid across window (175), similar to the laminar flow provided by lumen (240) as described above. This may be attributed to the same reasons as those provided above with respect to lumen (240). In addition or in the alternative, laminar flow may be promoted by suction from second lumen (342). For instance, the mere presence of suction may promote laminar flow and/or otherwise enhance the washing effects provided for window (175). The positioning of the distal termination point of second lumen (342) may also promote laminar flow and/or otherwise enhance the washing effects provided for window (175). As shown, the distal termination point of second lumen (342) is distal to the distal termination point of first lumen (340); yet proximal to the distal termination point of the underside of shaft (130) distal end (170). As can be seen in FIG. 10, the distal end configuration of shaft (230) is substantially similar to the configuration of distal end (170). This complementary configuration may also promote laminar flow and/or otherwise enhance the washing effects provided for window (175).

In some other versions, suction source (384) is replaced with a second fluid source, such that combinations of various fluids may be communicated through first fluid lumen (340) and second fluid lumen (342). In some such versions, first and second lumens (340, 342) deliver the same type of fluid. For instance, saline may be provided through first and second fluid lumens (340, 342) or pressurized gas may be provided through first and second fluid lumens (340, 342). In some other versions, first and second lumens (340, 342) may deliver different types of fluids, such that first and second lumens (340, 342) may provide any combination of saline (or other liquid) and/or pressurized gas. As yet another merely illustrative variation, first and second lumens (340, 342) may both provide suction. Furthermore, one or both of first or second lumens (340, 342) may alternate between providing fluid and suction.

In some versions, first and second lumens (340, 342) may be completely defined within shaft (330) of washing assembly (310) instead of being defined between shaft (130) and shaft (330). For instance, FIGS. 15-16 show an exemplary shaft (430) that may be incorporated into washing assemblies (210, 310) with first and second lumens (440, 442) terminating at a distal end (432). As shown in FIG. 16, shaft (430) comprises a circular opening (433). A first lumen (440) is located on a top portion of shaft (430) and extends less than half way around shaft (430). A second lumen (442) is located on a bottom portion of shaft (430) and extends less than half way around shaft (430). First and second lumens (440, 442) may be uniformly sized, or one of lumens (440, 442) may be larger than the other lumen (440, 442). For instance, first lumen (440) may extend more than halfway around shaft (430) if second lumen (442) is smaller in size; or second lumen (442) may extend more than halfway around shaft (430) if first lumen (440) is smaller in size. Other fluid lumen (440, 442) configurations will be apparent to one with ordinary skill in the art in view of the teachings herein. It should be understood that lumens (440, 442) may provide the same functionality as lumens (340, 342) described above. Lumens (440, 442) may be formed in numerous ways, including through a process of extruding shaft (430); a process of molding shaft (430); a process of securing two concentric tubes together (e.g. an outer tube to form shaft (430) and an inner tube to form opening (433)) with shims between the tubes to form lumens (340, 342), etc. It should also be understood that lumens (440, 442) may extend along the full length of shaft (430) or just along a portion of the length of shaft (430) (e.g. just along a distal portion). Lumens (440, 442) may be fluidly isolated from each other (e.g., in versions where lumens (440, 442) extend along the full length of shaft (430)); or may be in fluid communication with each other (e.g., in versions where lumens (440, 442) extend along only part of the length of shaft (430)).

B. Exemplary Actuation Assemblies

It may be desirable to selectively activate the flow of fluid through washing assemblies (210, 310) to selectively clear debris from distal end (170) of endoscope (110) with an actuation assembly. The actuation assembly may be internally incorporated into washing assemblies (210, 310) or the actuation assembly may be remotely incorporated into washing assemblies (210, 310). The examples below include merely illustrative versions of actuation assemblies that may be readily incorporated to a variation of washing assemblies (210, 310).

1. Exemplary Internal Actuation Assembly

FIGS. 17A-17B show an exemplary actuation assembly that may be readily incorporated into hubs (270, 370) of washing assemblies (210, 310). Actuation assembly (510) comprises an inner member (600) positioned within an outer member (500). Inner member (600) comprises a passageway (604) and a port (602) extending transversely to passageway (604). Outer member (500) comprises a passageway (504) coaxially aligned with passageway (604) and a transverse port (502) extending inwardly through outer member (500). Shaft (130) of endoscope (110) may be received through passageways (604, 504) and may be longitudinally secured to inner member (600). As another merely illustrative example, inner member (600) may be provided as a feature of a fixed mount (272, 372); or as a feature of a shaft (230, 330, 430). Port (502) is coupled to a fluid source such as fluid source (382). Inner member (600) is configured to translate relative to outer member (500) and to thereby act as a translating valve. A resilient member (608) is positioned between a distal end of inner member (600) and an inner wall (506) of outer member (500). Resilient member (608) biases outer member (500) distally to a closed position. O-rings (606) are positioned between inner member (600) and outer member (500), on each side port (502) and at the proximal end of outer member (500), to fluidly seal inner member (600) and outer member (500).

FIG. 17A shows actuation assembly (510) in a closed position. Resilient member (608) pushes outer member (500) to a distal position relative to inner member (600). In the distal position, port (602) of inner member (600) is positioned at a different longitudinal position than port (502) of outer member such that inner member (600) does not receive fluid through port (502). O-rings (606) isolate port (502) and passageway (504) from each other in this state. FIG. 17B shows actuation assembly (510) in an open position. Outer member (500) is translated to a proximal position relative to inner member (600) to longitudinally align port (602) with port (502). Accordingly, fluid provided to port (502) is communicated to port (602) of inner member (600). The fluid may then be provided to distal end (170) of endoscope (110) for washing as described above. Outer member (500) may coaxially be positioned around inner member (600) and shaft (130) such that a user may grasp outer member (500) to translate outer member (500). Alternatively, a lever may be provided with hub (270, 370) such that the lever is configured to translate outer member (500). Other suitable outer member (500) configurations will be apparent to one with ordinary skill in the art in view of the teachings herein.

2. Exemplary Remote Actuation Assembly

FIGS. 18-22B show an exemplary remote actuation assembly (710) that may be readily incorporated into washing assembly (210) to selectively control the fluid to wash distal end (170) of endoscope (110). As shown in FIG. 18, actuation assembly (710) comprises a set of tubes (702, 704, 706), a y-fitting (708), and bulb (712). Tube (706) couples a fluid source (700) to y-fitting (708). Tube (704) couples bulb (712) to y-fitting (708). Y-fitting (708) is then coupled to fluid inlet (274) of fixed mount (272) via tube (702). Bulb (712) may be squeezed multiple times to provide suction to distal end (170). Alternatively, bulb (712) may be actuated to communicate the fluid stored in bulb (712) to fixed mount (272) without activating fluid source (700). This may be desirable when a user only needs a small amount of fluid to rinse distal end (170) of endoscope (110). Bulb (712) may be handheld to actuate bulb (712) with an operator's hand. In some versions, bulb (712) is positioned around the axis of hub (270), such that the operator may squeeze bulb (712) using the same hand that grasps endoscope (110) and/or washing assembly (210).

FIG. 19 shows a merely illustrative variation of remote actuation assembly (710) where bulb (712) may be placed within a foot pedal (800) to actuate bulb (712) with a foot. A foot pedal (800) may be desirable to allow a surgeon to use his/her hands for other tasks, such as positioning endoscope (110) or dilation catheter system (1). Foot pedal (800) may be reusable or disposable. Foot pedal (800) of the present example comprises a top plate (802) and a bottom plate (804) positioned under top plate (802). Top plate (802) comprises an extension (810) extending downwardly and bottom plate (804) comprises an extension (808) extending upwardly. Extensions (808, 810) are coupled via pin (809) such that top plate (802) is pivotable relative to bottom plate (804). A torsion spring (not shown) resiliently biases top plate (802) relative to bottom plate (804) to the non-actuated position shown in FIGS. 20A, 21A, and 22A.

Bulb (712) and fluid source tube (706) are placed between top plate (802) and bottom plate (804) as shown in FIGS. 19-22B. In particular, as best seen in FIGS. 20A-21B, bulb (712) is positioned within one side of foot pedal (800). This side of bottom plate (804) comprises a pair of retention members (805) extending upwardly from bottom plate (804). Retention members (805) are used to retain bulb (712) within bottom plate (804). Any suitable number of retention members (805) may be used. It should also be understood that retention members (805) may restrict the degree to which top plate (802) may be pivoted relative to bottom plate (804) when top plate is pivoted to an actuated position. The opposing side of top plate (802) comprises a block (803) extending downwardly toward bottom plate (804). Block (803) is positioned between retention members (805). When a user steps on top plate (802) to provide fluid from fluid source (700) (FIGS. 20B, 21B, 22B), block (803) of top plate (802) engages bulb (712) to compress bulb (712) and thereby prepare bulb (712) for suction by evacuating air from and/or liquid bulb (712).

As best seen in FIGS. 20A-20B and 22A-22B, tube (706) is positioned in the opposite side of foot pedal (800). Bottom plate (804) comprises retention members (814) and protrusion (816) positioned between retention members (814). Retention members (814) extend upwardly toward top plate (802) and are configured to hold tube (706) within foot pedal (800). Any suitable number of retention members (814) may be used. Protrusion (816) extends upwardly from bottom plate (804) toward top plate (802) and comprises a recess (818). Top plate (802) comprises a block (810) positioned above protrusion (816). Block (810) comprises a pointed tip (812) corresponding to recess (818). When top plate (802) is in a first position, pointed tip (812) pushes tube (706) against recess (818) to close tube (706) as shown in FIG. 20A. Pointed tip (812) and protrusion (816) thus cooperate to provide a pinch valve. This prevents fluid from fluid source (700) from being communicated to washing assembly (210). It should be understood that pointed tip (812) does not pierce tube (706) when tube (706) is pinched between pointed tip (812) and recess (818) of protrusion (816). When a user steps on top plate (802) to move top plate (802) to an actuated position, pointed tip (812) is released from tube (706) and recess (818) as shown in FIGS. 20B and 22B. This allows fluid to be communicated from fluid source (700) to washing assembly (210) via tube (706). In some versions, fluid source (700) is located at a higher vertical position than washing assembly (210), such that gravity drives fluid from fluid source (700) to washing assembly (210). In some other versions, fluid source (700) is pressurized.

It should be understood from the foregoing that bulb (712) is compressed as shown in FIGS. 20B and 21B when fluid communication is opened through tube (706) as shown in FIGS. 20B and 22B. When a user releases top plate (802) from the actuated position to stop providing fluid from fluid source (700) (FIGS. 20A and 22A), the torsion spring resiliently pivots top plate (802) back to the non-actuated position such that block (803) of top plate (802) disengages bulb (712) as shown in FIGS. 20B and 21B. When block (803) disengages bulb (712), bulb (712) expands to draw air and/or fluid into bulb (712) from distal end (170) of endoscope (110), thereby providing suction at distal end (170). Thus, when foot pedal (800) is transitioned to the actuated state, saline or some other fluid is communicated via tube (702) to the distal end (170) of endoscope (110); and suction is communicated via tube (702) to the distal end (170) of endoscope (110) when foot pedal (800) transitions back to the non-actuated state. This process may be repeated as many times as desired to wash window (175) and/or to flush the site within the patient.

In some versions, it may be desirable to actuate bulb (712) without simultaneously communicating fluid from fluid source (700) to distal end (170). For instance, an operator may wish to simply create suction at distal end (170) to clear excess fluid from the endoscope (110) field. Accordingly, another exemplary foot pedal (900) is shown in FIGS. 23A-23B in which bulb (712) may be actuated independently from fluid source (700). Foot pedal (900) is similar to foot pedal (800), except that foot pedal (900) comprises a second top plate (910) to independently actuate bulb (712). Bulb (712) is placed within foot pedal (900) such that retention members (905) of bottom plate (904) hold bulb (712) within foot pedal (900). Bottom plate (904) is similar to bottom plate (804) except that bottom plate (904) may be wider to accommodate second top plate (910). Top plate (902) is similar to top plate (802) and comprises block (903) to engage bulb (712) and torsion spring (not shown) to resiliently bias top plate (802) relative to bottom plate (904). Top plate (902) may be actuated in the same way as top plate (802) to simultaneously actuate bulb (712) and fluid source (700). Second top plate (910) is positioned adjacent to top plate (902). Second top plate (910) comprises a block (914) extending downwardly toward bottom plate (904). Second top plate (910) is biased to a first position by a torsion spring (not shown) positioned between second top plate (910) and bottom plate (904) such that block (914) is positioned over bulb (712), as shown in FIG. 22A.

A user may independently actuate bulb (712) by stepping on second top plate (910) independently of top plate (902) to move second top plate (910) to an actuated position, as shown in FIG. 23B. In the actuated position, block (914) of second top plate (910) engages bulb (712) to compress bulb (712) and evacuate air from bulb (712). Second top plate (910) may then be released to move second top plate (910) back to the non-actuated position (FIG. 23A). Bulb (712) is then decompressed and expands to draw fluid back into bulb (712) from distal end (170) of endoscope (110) to provide suction at distal end (170). This process of independently actuating second top plate (910) may be repeated as many times as desired to continue providing suction at distal end (170) of endoscope (110).

3. Exemplary Button Valve Assembly

FIG. 24 depicts yet another system that may be coupled with washing assembly (120) to wash distal end (170) of endoscope. In particular, endoscope (110) is fitted with a button valve assembly (1000) comprising a grip body (1002) that is releasably secured to handle (152) of endoscope (110) by a set of clip arms (1004). Grip body (1002) is configured to rest in an operator's palm when the operator grasps endoscope (110) and body (1002) with a palmar grasp, with the thumb of the operator's grasping hand pointing generally toward the proximal end of endoscope (110). Button valve assembly (1000) further includes a pair of valve buttons (1020, 1030). While valve buttons (1020, 1030) are shown as being integrated within grip body (1000), it should be understood that valve buttons (1020, 1030) may instead be positioned at any other suitable locations. By way of example only, valve buttons (1020, 1030) may be positioned proximal to grip body (1000) (e.g., just lateral to eyepiece (150), etc.). Other suitable locations for valve buttons (1020) will be apparent to those of ordinary skill in the art in view of the teachings herein. In the present example, valve buttons (1020, 1030) are positioned and oriented such that the operator may independently actuate each valve button (1020, 1030) using the thumb of the same hand that is grasping endoscope (110) and body (1002). In particular, the operator may actuate each valve button (1020, 1030) by using the thumb of their grasping hand to depress each valve button (1020, 1030) along a path that is transverse to the longitudinal axis of shaft (130).

Valve button (1020) is operable to selectively provide a fluid (e.g., saline, pressurized air, etc.) to shaft (230), to thereby selectively wash at least a portion of distal end (170) of shaft (130) (e.g., at least window (175)). In particular, valve button (1020) is coupled with a fluid source (1024) via a conduit (1022). Valve button (1020) is further coupled with a y-fitting (1070). Y-fitting (1070) is coupled with a conduit (1074), which is further coupled with fluid inlet (274) of mount (272). Valve button (1020) is operable to selectively open and close the fluid path between conduit (1022) and conduit (1074); and is thereby operable to selectively open and close the fluid path between fluid source (1024) and the distal end (170) of shaft (130). By way of example only, valve button (1020) may actuate a conventional translating valve that is oriented along a path generally transverse to the longitudinal axis of shaft (130). Various suitable types of valves that may be actuated by valve button (1020) will be apparent to those of ordinary skill in the art in view of the teachings herein. Similarly, various suitable forms that fluid source (1024) may take will be apparent to those of ordinary skill in the art in view of the teachings herein.

Valve button (1030) is operable to selectively provide suction to shaft (230), to thereby draw fluid away from the side adjacent to distal end (170) of shaft (130). In particular, valve button (1030) is coupled with a suction source (1034) via a conduit (1032). Valve button (1030) is further coupled with y-fitting (1070). As noted above, y-fitting (1070) is coupled with conduit (1074), which is further coupled with fluid inlet (274) of mount (272). Valve button (1030) is operable to selectively open and close the fluid path between conduit (1032) and conduit (1074); and is thereby operable to selectively open and close the fluid path between suction source (1034) and the distal end (170) of shaft (130). By way of example only, valve button (1030) may actuate a conventional translating valve that is oriented along a path generally transverse to the longitudinal axis of shaft (130). Various suitable types of valves that may be actuated by valve button (1030) will be apparent to those of ordinary skill in the art in view of the teachings herein. Similarly, various suitable forms that suction source (1034) may take will be apparent to those of ordinary skill in the art in view of the teachings herein.

In some versions, at least part of the fluid paths provided by button valve assembly (1000) (e.g., conduits (1022, 1032, 1074), y-fitting (1070), etc.) are defined by lumens formed in body (1002). In addition or in the alternative, such fluid paths may be provided by conventional tubing and/or other components. In such versions, such conventional components may be inserted in body (1002), retained in recesses formed in body (1002), clipped to body (1002), or have any other suitable relationship with body (1002). It should also be understood that body (1002) may be varied in numerous ways. By way of example only, body (1002) may be positioned on the opposite side of handle (152), beneath light post (109) such that light post (109) is generally positioned in the crook between the thumb and index finger of the hand grasping endoscope (110). Body (1002) may include a shield feature (not shown) that would be positioned between the hand of the operator and light post (109), to thereby substantially isolate the hand of the operator from heat emanated by light post (109). Valve buttons (1020, 1030) may be located at a position that is lateral to handle (152), where valve buttons (1020, 1030) may be readily depressed by the thumb of the hand grasping endoscope (110). In any of the arrangements described above, the operator may still readily operate dial (108) as well, without having to substantially reposition their grasping hand. In other words, the operator may easily transition between actuation of buttons (1020, 1030) and actuation of dial (108). Body (1002) may also come in different sizes and configurations selectable based on the hand size of the operator, the operator's preference, etc. Other suitable configurations for body (1002) and positions for buttons (1020, 1030) will be apparent to those of ordinary skill in the art in view of the teachings herein.

It should be understood from the foregoing that an operator may readily provide either fluid or suction to distal end (170) of shaft (130) with the same hand that grasps endoscope (110), and without having to reposition the grasping hand from a normal operating position such that dial (108) remains easily accessible. It should also be understood that the operator may depress button (1020) as long as desired to provide fluid to distal end (170) of shaft (130); or button (1030) as long as desired to provide suction to distal end (170) of shaft (130). Button valve assembly (1000) may be used with any of the various shafts (230, 330, 430) described herein or any other suitable type of shaft. Similarly, button valve assembly (1000) may be used with any suitable endoscope, and is not limited in use to endoscope (110) described herein.

IV. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A washing system, wherein the washing system is operable to wash an endoscope, wherein the washing system comprises:
(a) a hub configured to couple with a fluid source; and
(b) a shaft extending distally from the hub, wherein the shaft comprises:
(i) a distal portion, wherein the distal portion of the washing assembly is configured to correspond with a distal portion of an endoscope,
(ii) a first fluid channel extending from the hub to the distal portion, wherein the first fluid channel is configured to provide fluid communication between the hub and the distal portion,
(iii) a lumen extending through the shaft, wherein the lumen is configured to receive the endoscope, and
(iv) a boss structure positioned within the shaft, wherein the boss structure is configured to maintain a radial position of an endoscope relative to the first fluid channel, wherein the boss structure extends along at least part of the length of the shaft.

2. The washing system of claim 1, wherein the hub comprises an inner member and an outer member, wherein the outer member is configured to couple with a fluid source, wherein the inner member of the hub is configured to rotate relative to the outer member of the hub.

3. The washing system of claim 1, further comprising an endoscope having a shaft, wherein the shaft is inserted in the lumen, wherein the first fluid channel is positioned between the shaft of the washing assembly and the shaft of the endoscope.

4. The washing system of claim 3, wherein the lumen of the shaft of the washing assembly includes an inner surface, wherein the shaft of the endoscope includes an outer surface, wherein the first fluid channel is defined by a combination of:
(A) the inner surface of the shaft of the washing assembly,
(B) the outer surface of the shaft of the endoscope, and
(C) the boss structure.

5. The washing system of claim 1, wherein the boss structure comprises at least one ridge extending into the lumen, wherein the ridge is configured to define at least a portion of the first fluid channel.

6. The washing system of claim 1, wherein the first fluid channel comprises a discrete lumen formed within the shaft.

7. The washing system of claim 1, further comprising a fluid source configured to be coupled with the washing assembly, wherein the fluid source is configured to provide saline to the washing assembly.

8. The washing system of claim 1, further comprising a fluid source configured to be coupled with the washing assembly, wherein the fluid source is configured to provide suction to the washing assembly.

9. The washing system of claim 1, further comprising a fluid source configured to be coupled with the washing assembly, wherein the fluid source is configured to provide pressurized gas to the washing assembly.

10. The washing system of claim 1, wherein the shaft further comprises a second fluid channel, wherein the first fluid channel is configured to couple with a first fluid source, wherein the second fluid channel is configured to couple with a second fluid source.

11. The washing system of claim 10, wherein the first and second fluid channels are fluidly isolated relative to each other along the length of the shaft.

12. The washing system of claim 1, wherein the fluid channel is configured to provide a laminar flow of fluid across a window of an endoscope inserted in the lumen.

13. The washing system of claim 1, further comprising an actuation assembly configured to selectively actuate a fluid source, wherein the actuation assembly comprises an inner member and an outer member, wherein the outer member is configured to translate relative to the inner member to selectively control fluid communication to the first fluid channel.

14. The washing system of claim 1, further comprising an actuation assembly configured to selectively actuate a fluid source, wherein the actuation assembly comprises a bulb positioned between the fluid source and the washing assembly, wherein the bulb is configured to provide suction to the washing assembly.

15. The washing system of claim 14, wherein the actuation assembly comprises a foot pedal.

16. The washing system of claim 15, wherein the foot pedal is configured to simultaneously actuate the fluid source and the bulb.

17. The washing system of claim 15, wherein the foot pedal is configured to independently actuate the fluid source and the bulb.

18. The washing system of claim 1, further comprising a valve assembly adjacent to the hub, wherein the valve assembly comprises:
(i) an outlet conduit coupled with the first fluid channel,
(ii) a first inlet configured to couple with a first source,
(iii) a second inlet configured to couple with a second source,
(iv) a first valve having an actuator, wherein the actuator of the first valve is operable to selectively couple the first inlet with the outlet conduit, and
(v) a second valve having an actuator, wherein the actuator of the second valve is operable to selectively couple the second inlet with the outlet conduit.

19. A washing system, wherein the washing system is operable to wash an endoscope, wherein the washing system comprises:
(a) a hub configured to couple with a fluid source; and
(b) a shaft extending distally from the hub, wherein the shaft comprises:
(i) a distal portion, wherein the distal portion of the washing assembly is configured to correspond with a distal portion of an endoscope,
(ii) a first fluid channel extending from the hub to the distal portion, wherein the fluid channel is configured to provide fluid communication between the hub and the distal portion,
(iii) a second fluid channel extending from the hub to the distal portion, wherein the second fluid channel is configured to provide suction communication to the distal portion, and
(iv) a lumen extending through the shaft, wherein the lumen is configured to receive the endoscope, wherein the lumen defines a longitudinal axis,
wherein the first and second fluid channels are positioned on opposite transverse sides of the longitudinal axis.

20. A washing system, wherein the washing system is operable to wash an endoscope, wherein the washing system comprises:
(a) a hub; and
(b) a shaft extending distally from the hub, wherein the shaft comprises:
(i) a distal portion, wherein the distal portion of the washing assembly is configured to longitudinally align with a distal portion of an endoscope,
(ii) at least one fluid channel extending from the hub to the distal portion, wherein the at least one fluid channel is configured to provide fluid communication between the hub and the distal portion, and
(iii) a lumen extending through the shaft, wherein the lumen is configured to receive the endoscope;
(c) a fluid communication assembly coupled with the hub, wherein the fluid communication assembly is operable to selectively communicate fluid and suction to the at least one fluid channel via the hub.
